(19) United States
(12) Patent Application Publication
(10) Pub. No.: US 2026/0345106 A1
(43) Pub. Date: Nov. 5, 2026

(54) SUPERELLIPSE SETS FOR MYOCARDIAL IMAGING

(71) Applicant: Siemens Medical Solutions USA, Inc., Malvern, PA (US)

(72) Inventors: Maximilian Reymann, Erlangen (DE); Alexander Hans Vija, Evanston, IL (US)

(21) Appl. No.: 19/558,572

(22) Filed: Mar. 6, 2026

Related U.S. Application Data
(60) Provisional application No. 63/798,715, filed on May 2, 2025.

(51) Int. Cl. A61B 6/00; A61B 6/50

(52) U.S. Cl. CPC A61B 6/5223; A61B 6/503; A61B 6/5217

(57) ABSTRACT

A system and method include acquisition of a three-dimensional image of an anatomical structure, determination of a plurality of slices of the three-dimensional image, for each of the plurality of slices, fitting of a model comprising a plurality of parameters to the anatomical structure in the slice to determine values of each of the plurality of parameters; and determination of a characterization of the anatomical structure based on the values of each of the plurality of parameters determined for each of the plurality of slices.

### CROSS-REFERENCE TO RELATED APPLICATIONS

This application claims priority to, and the benefit of, U.S. Provisional Patent Application Ser. No. 63/798,715, filed May 2, 2025, the contents of which are incorporated herein by reference for all purposes.

### BACKGROUND

Cardiovascular diseases are a leading cause of morbidity and mortality. These diseases are often associated with structural abnormalities of the heart. Modern medical imaging modalities (e.g., echocardiography, computed tomography (CT), single-photon emission computed tomography (SPECT), positron emission tomography (PET) and magnetic resonance imaging (MRI)) are capable of acquiring detailed images of the heart. These images may be used for diagnosis and treatment planning.

Due to their clinical importance, the images should be as accurate and artifact-free as possible. Corrections are therefore applied to the images to account for the effects of respiratory motion and modality-specific imaging characteristics. The quality of these corrections is conventionally assessed through reader studies, in which multiple experts manually compare images which are reconstructed with various corrections (including no correction) applied. Reader studies are time consuming, expensive and subjective.

Accordingly, there is a need for systems and methods to efficiently assess different corrections applied to a same set of image data. Such assessments are preferably reproducible, quantitative, and not reliant on subjective opinion. Efficient assessments of image corrections may provide improved images and lead to improved diagnoses, monitoring of disease progression, and evaluation of therapeutic interventions.

### BRIEF DESCRIPTION OF THE DRAWINGS

The foregoing and other aspects of some embodiments are best understood from the following detailed description when read in connection with the accompanying drawings. Embodiments are not limited to the specific implementations disclosed herein.

FIG. 1 is a block diagram of a system to characterize the shape of an anatomical structure using superellipse parameters according to some embodiments;

FIGS. 2A and 2B comprise a flow diagram of a process to characterize the shape of an anatomical structure using superellipse parameters according to some embodiments;

FIG. 3 is a slice image of a portion of a left ventricle according to some embodiments;

FIG. 4 illustrates a line through a center of the portion of the left ventricle of the FIG. 3 image according to some embodiments;

FIG. 5 is a profile of pixel intensities along the line of FIG. 4 according to some embodiments;

FIG. 6A illustrates a Gaussian function fit to a first peak of the FIG. 5 intensity profile according to some embodiments;

FIG. 6B illustrates a Gaussian function fit to a second peak of the FIG. 5 intensity profile according to some embodiments;

FIG. 7 illustrates locations of peak intensities along the line of FIG. 4 according to some embodiments;

FIG. 8 illustrates locations of peak intensities along several lines through the center of the portion of the left ventricle of the FIG. 3 image according to some embodiments;

FIG. 9 depicts a system to determine superellipse parameter values based on the peak intensities of FIG. 8 using a neural network according to some embodiments;

FIG. 10 depicts a system to characterize a region based on superellipse parameter values determined from a plurality of slice images according to some embodiments; and

FIG. 11 is a block diagram of an imaging system according to some embodiments.

### DETAILED DESCRIPTION

The following description is provided to enable any person in the art to make and use the described embodiments. Various modifications, however, will be readily-apparent to those in the art. Independent of the grammatical term usage, individuals with male, female or other gender identities are included within the term.

In cardiac images, the shape of the left ventricle can appear distorted due to imaging characteristics (e.g., respiratory motion and a slow rotating gamma camera). The present inventors have discovered that the shape of the left ventricle within an image may therefore act as a proxy for the accuracy of the image and the presence of artifacts in the image. The present inventors have further discovered a system to efficiently detect the left ventricle within an image and to quantify the shape of the left ventricle using a relatively small set of parameter values.

Accordingly, some embodiments efficiently characterize three-dimensional shapes of anatomical structures from three-dimensional images acquired by medical imaging systems. Generally, embodiments generate sets of superellipses fitted to an anatomical structure and the parameter values of each superellipse (e.g., semi-major axis (a), semi-minor axis (b), and exponent (n)) may be used to characterize the overall shape of the anatomical structure.

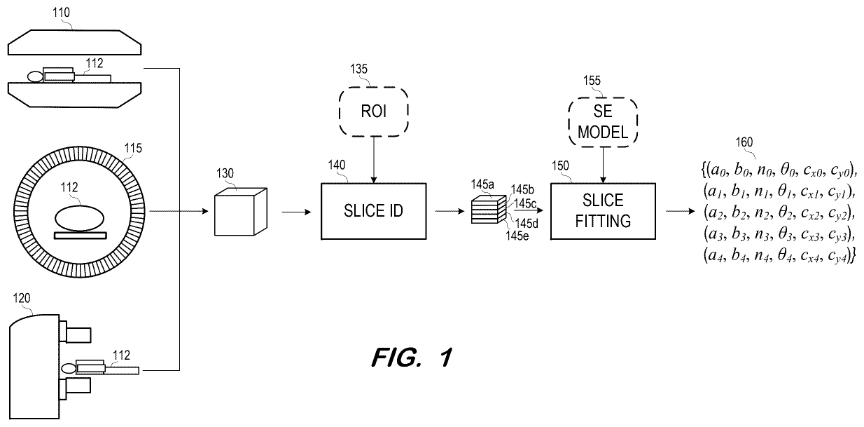

FIG. 1 is a block diagram of a system to characterize the shape of an anatomical structure using superellipse parameters according to some embodiments. Each component depicted in FIG. 1 may be implemented using any combination of hardware and/or software (i.e., executable program code), and one or more components may be implemented using the same hardware and/or software.

The anatomical structure is depicted in three-dimensional image 130, which is a three-dimensional set of pixel intensities representing an anatomical volume. Image 130 may be acquired/generated using any system that is or becomes known. As non-exhaustive examples, FIG. 1 indicates that image 130 may be an image of a volume of patient 112 which was generated using MR scanner 110, PET scanner 115 or SPECT scanner 120. In the case of scanners 115 and 120, image 130 may be generated by acquiring data and reconstructing the data based on a linear attenuation coefficient map of patient 112 acquired by, e.g., a CT scanner. Image 130 may undergo any suitable processing or corrections that are or become known, including but not limited to motion correction.

Region of interest 135 of image 130 is identified. For example, an operator may manipulate a user interface displaying image 130 to indicate a volume of interest which includes the anatomical structure. An automated or semi-automated segmentation algorithm may be executed to identify a predetermined region of interest 135. In the foregoing description, region of interest 135 includes a left ventricle depicted in image 130.

Slice identifier 140 may determine a three-dimensional sub-portion of image 130 which includes region of interest 135. In some cases, for example if region of interest 135 makes up a significant portion of image 130, slice identifier 140 may operate on entire image 130. Slice identifier 140 determines slices 145a-145e of the sub-portion of image 130.

Each of slices 145a-145e may be one voxel thick, but embodiments are not limited thereto. Embodiments are also not limited to five slices. A voxel according to some embodiments is a cubic region measuring 5 mm×5 mm×5 mm. Slices 145a-145e may be oriented such that the portion of the anatomical structure within each slice may be efficiently characterized by a superellipse. In a case that the region of interest is the left ventricle, slices 145a-145e may be oriented perpendicular to a major axis of the left ventricle.

Slice fitting component 150 fits the anatomical structure depicted in each slice 145a-145e to superellipse model 155. According to some embodiments, superellipse model 155 is as follows:

$${f}_{i} ( x , y ) = {\vert \frac{{x}_{i}^{′}}{{a}_{i}} \vert}^{{n}_{i}} + {\vert \frac{{y}_{i}^{′}}{{b}_{i}} \vert}^{{n}_{i}} - 1 , with$$

$${x}_{i}^{′} = ( x - {c}_{x , i} ) cos {θ}_{i} + ( y - {c}_{y , i} ) sin {θ}_{i}$$

$${y}_{i}^{′} = - ( x - {c}_{x , i} ) sin {θ}_{i} + ( y - {c}_{y , i} ) cos {θ}_{i}$$

Accordingly, slice fitting component 150 determines values 160 for parameters α, b, n, θ, cₓ, c_{y} for each slice i. α represents a length of a semi-major axis, b represents a length of a semi-minor axis, n represents a squareness, or curvature, θ represents a rotation, and cₓ, c_{y} are coordinates of the center. Parameter values 160 advantageously provide a succinct, accurate and reproducible characterization of the three-dimensional anatomical structure. Several methods for determining parameter values 160 will be described below.

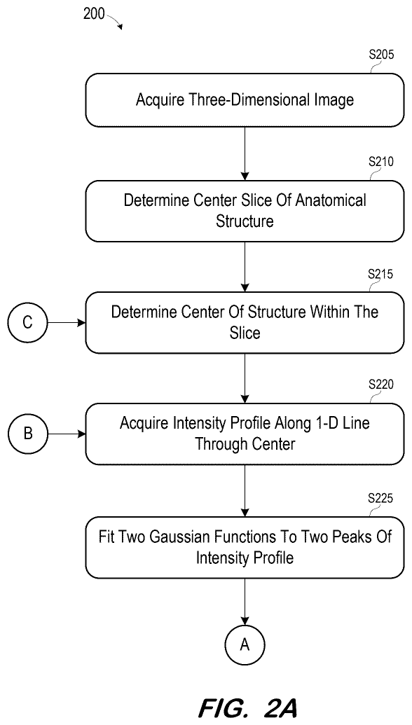

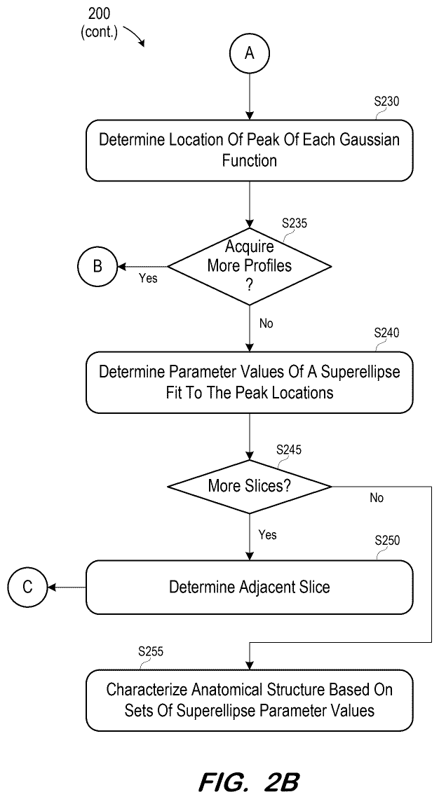

FIGS. 2A and 2B comprise a flow diagram of process 200 to characterize the shape of an anatomical structure using superellipse parameters according to some embodiments. Process 200 may be performed by any combination of hardware and software that is or becomes known, and need not be performed by an imaging system. Program code embodying these processes may be stored by any non-transitory tangible medium, including a fixed disk, a volatile or non-volatile random-access memory, a DVD, a Flash drive, and a magnetic tape, and executed by any suitable processing unit, including but not limited to one or more microprocessors, microcontrollers, processor cores, and processor threads. Embodiments are not limited to the examples described below.

Initially, a three-dimensional image is acquired at S205. The three-dimensional image may be acquired by an imaging system or from a computer-readable storage medium. For example, the three-dimensional image may have been generated days, months or years prior to S205 and in a different location. The three-dimensional image consists of voxels (which will also be referred to herein as pixels), each of which is assigned an intensity value. The intensity value at a voxel may represent an anatomical structure located at the voxel as is known in the art.

A center slice of the anatomical structure is determined at S210. The center point of the anatomical structure (e.g., the left ventricle) may be determined from the three-dimensional image using any method or approximation that is or becomes known. The determined central slice is the slice of the image which includes the center point. As described above, the center slice may be perpendicular to the major axis of the left ventricle.

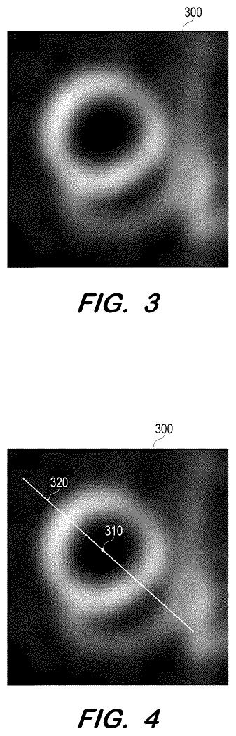

FIG. 3 shows axial slice 300 determined at S210 according to one example. The bright ring shown in slice 300 depicts a portion of the myocardial wall which resides in slice 300. A center of the anatomical structure within the slice is determined at S215. In the first iteration of S215, the center of the anatomical structure within the slice is the center point determined at S210.

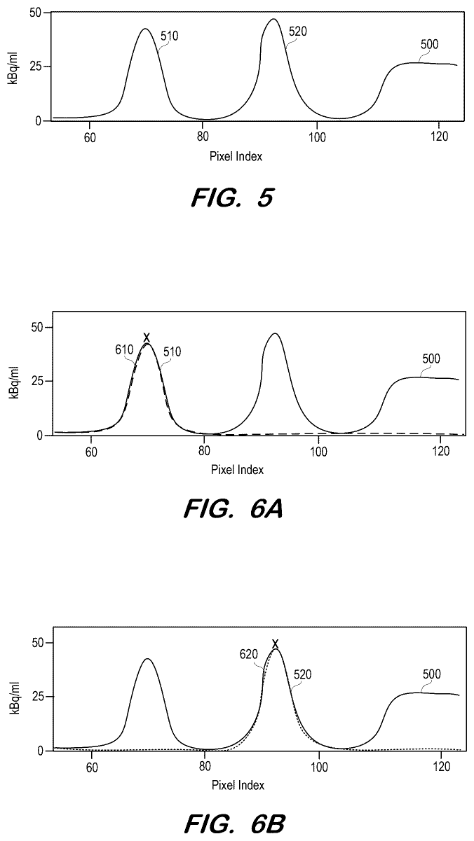

At S220, an intensity profile along a one-dimensional line through the center of the region is acquired. FIG. 4 shows line 320 through center 310 of the region according to some embodiments. S220 consists of determining the intensities of all pixels along line 320. FIG. 5 illustrates acquired profile 500 of the pixel intensities along line 320 according to some embodiments. As shown, profile 500 includes peaks 510 and 520 which correspond to locations of the myocardial wall within image 300.

Next, at S225, two Gaussian functions are fit to two peaks of the intensity profile. S225 may include detection of two peaks of the profile and determination of a window of intensities about each peak to which the parameters of a Gaussian function will be fitted. FIG. 6A illustrates function 610 fit to peak 510 of profile 500 according to some embodiments, and FIG. 6B illustrates function 620 fit to peak 520 of profile 500 according to some embodiments.

The intensity profile may include three or more peaks of similar magnitude, so S225 may include determining the two of the three or more peaks to which Gaussian functions will be fitted. For example, S225 may consider only peaks located within a radius of 4 cm around the center of the region of interest determined at S215. In some embodiments, S225 identifies the two peaks with the smallest distance to the center in order to avoid identifying additional peaks that might exist around the left ventricle.

Due to the many possible combinations of peaks and intensities of the intensity profile, some embodiments implement a robust algorithm for locating two peaks which represent the myocardial wall, locating windows about the peaks to which the Gaussian functions will be fit, and fitting the Gaussian functions. Embodiments are not limited to the foregoing algorithm.

Initially, all peaks are identified which have a minimal distance of 1 cm (assuming the diameter of the left ventricle is ≥1 cm), a peak intensity value of at least 20% of the maximum intensity value of the profile and a minimum width of one pixel. Also, all minima along the profile that have a minimal distance of 1 cm and a minimum width of 1 pixel are identified.

Next, a window of pixels is determined for each peak of the profile. Starting from the left-most identified peak, the closest minimum strictly to the left and strictly to the right (i.e., the nearest flanking minima) are identified. If no minimum exists on a side of the peak, the nearest profile boundary (i.e., pixel index 0 or the last pixel index) is identified. A symmetric window is determined which is centered on the peak and which fits entirely between the two minima which were determined for the peak. The intensity values within this symmetric window are determined and these will be the values to which a Gaussian function will be fit.

The Gaussian fit to each peak may consist of an optimization which is constrained by initialization values: amplitude=intensity value at the peak, center=pixel index at the peak, width=window width h if h<3 cm, otherwise use 3 cm. During the optimization, the amplitude may be constrained to ±1% around the observed peak intensity value, the center limited to the symmetric window, and the width bounded to [half a pixel size, and 3 cm].

According to some embodiments, a weighted least squares using a Gaussian weighting is applied that emphasizes samples near the peak center (i.e., samples near the center are weighted more heavily than sample near the window edges: weight

$${r}_{i} = exp ( - \frac{{( {x}_{i} - p )}^{2}}{h / {2}^{2}} ) ,$$

where h is the current window width and p is the peak pixel index. The weighted least-squares objective minimized by the fit may be:

$$J ( A , μ , σ ) = {\sum }_{i ∈ 𝒲} {{r}_{i}^{2} [ {y}_{i} - G ( {x}_{i} ; A , μ , σ ) ]}^{2}$$

where [symbol] indexes the window samples, A is the amplitude, μ is the mean/center, σ is the gaussian width, and G is a Gaussian function.

The above process results in a Gaussian fit for all peaks found on the profile. Since the number of peaks may be greater than two, the fitted peaks are post-processed to identify two of the peaks representing the myocardial wall. For example, peaks within 0.6 cm of the profile midpoint may be excluded since they are likely caused by blood-pool activity. From the remaining peaks, the two peaks closest to the profile center are selected.

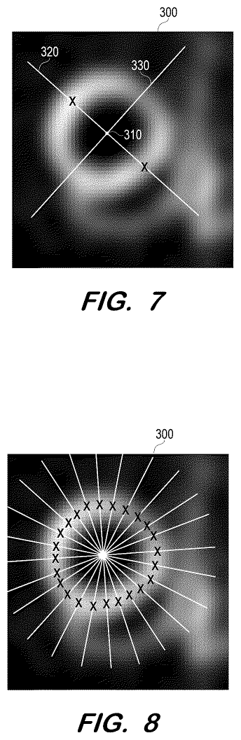

Returning to process 200, the locations of the peaks of each of the two Gaussian functions are determined at S230. Deriving the peak locations from the Gaussian functions allows sub-pixel resolution. These locations are assumed to be the (x, y) coordinates of the myocardial wall along the profile. The locations are denoted by “X” in FIGS. 6A and 6B, and corresponding locations of image 300 are denoted by “X” along line 320 of FIG. 7.

At S235 it is determined whether more intensity profiles are to be acquired. It will be assumed, for example, that twelve intensity profiles are to be acquired. Accordingly, flow returns to S220 to acquire intensity profile along another line through the center of the region of interest. FIG. 7 shows line 330 along which the intensity profile may be acquired.

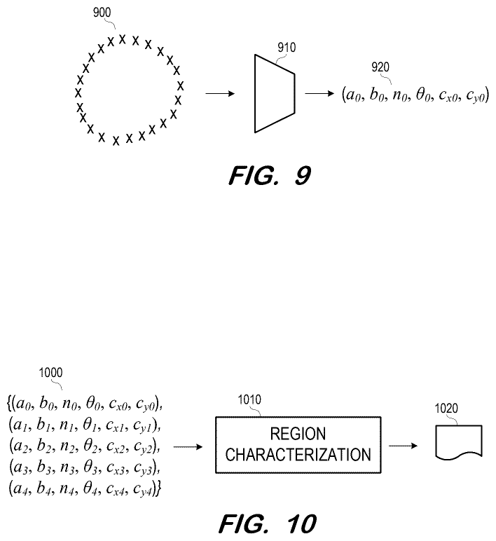

Flow cycles between S220 and S235 until two peak locations have been determined for the desired number of intensity profiles. FIG. 8 shows peak locations determined for each of twelve one-dimensional lines according to some embodiments. At S240, a superellipse is fit to the peak locations. Fitting a superellipse comprises determining values of parameters (α, b, n, θ, cₓ, c_{y}) of a superellipse based on the peak locations. S240 may include a non-linear least squares optimization regularized to physiological bounds. FIG. 9 illustrates an embodiment in which peak locations 900 are input to neural network 910 to generate superellipse parameter values 920.

At S245, it is determined whether more slices of the anatomical structure exist. If so, another slice is determined at S250 and flow returns to S215. The determined slice may be adjacent to a slice which was already processed by process 200. Using an adjacent slice allows the known location of the center of an already-processed adjacent slice to be leveraged in the determination of the center of the slice at S215.

Flow therefore cycles between S215 and S250 to acquire a set of values of superellipse parameters for each of a plurality of slices. The sets of parameter values may constitute a comprehensive three-dimensional model of the anatomical structure. The model allows interpolation along all surface points to provide a detailed geometric representation. Accordingly, once it is determined at S245 that all slices have been processed, the anatomical structure may be characterized based on the sets of parameter values at S255.

FIG. 10 shows five sets 1000 of parameter values determined according to process 200. Region characterization component 1010 may receive sets 1000 and output characterization 1020. Characterization 1020 may comprise any finding which may be determined based on sets 1000. For example, characterization 1020 may comprise an indication of abnormality, i.e., a deviance from an expected “normal” shape which is greater than a threshold. In the case of the left ventricle, the fit superellipses are expected to be relatively circular (α=b and n=2). Accordingly, characterization 1020 may indicate whether the superellipses deviate significantly from this expectation.

It is known that respiratory motion manifests as elongation of the imaged left ventricle in the superior-inferior direction. Therefore, characterization 1020 may indicate excessive motion during the acquisition of the image (and/or inadequate motion correction) if the fit superellipses are not sufficiently circular. In other examples, characterization 1010 may indicate one or more of a volume of the anatomical structure, a deviance from a “normal” volume, a degree of circularity along the major axis of the anatomical structure, a shape-based diagnosis, etc.

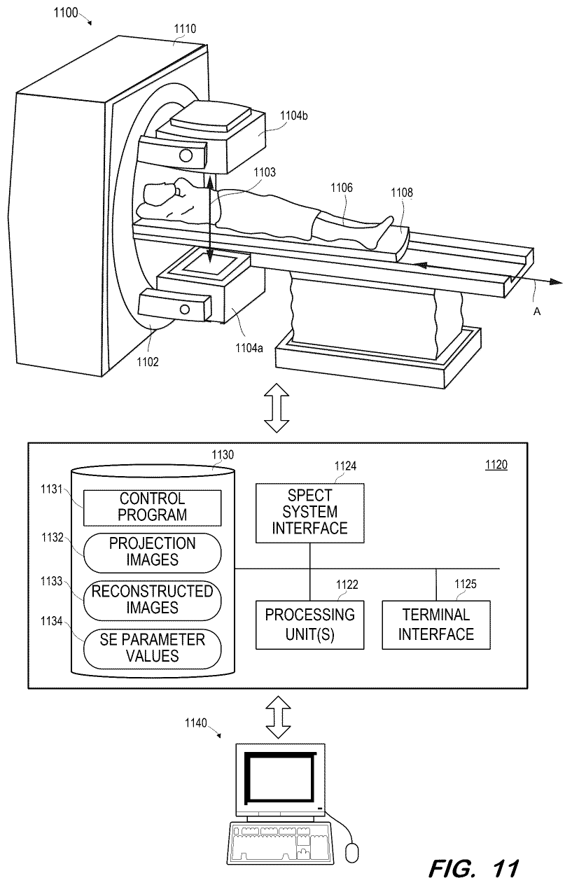

FIG. 11 illustrates imaging system 1100 for performing the processes described herein according to some embodiments. System 1100 is a SPECT imaging system as is known in the art, but embodiments are not limited thereto. As mentioned above, embodiments are not limited to an imaging system.

System 1100 includes housing 1110 for gantry 1102 to which two or more gamma cameras 1104a, 1104b are attached, although any number of gamma cameras can be used. A detector within each gamma camera detects gamma photons (i.e., emission data) emitted by a radioactive tracer injected into the body of patient 1106 lying on bed 1108. Bed 1108 is slidable along axis-of-motion A. At respective bed positions (i.e., imaging positions), a portion of the body of patient 1106 is positioned between gamma cameras 1104a, 1104b in order to capture emission data from that body portion from various projection angles.

Control system 1120 may comprise any general-purpose or dedicated computing system. Control system 1120 includes one or more processing units 1122 configured to execute executable program code to cause system 1120 to operate as described herein, and storage device 1130 for storing the program code. Storage device 1130 may comprise one or more fixed disks, solid-state random access memory, and/or removable media (e.g., a thumb drive) mounted in a corresponding interface (e.g., a USB port).

Storage device 1130 stores program code of control program 1131. One or more processing units 1122 may execute control program 1131 to, in conjunction with SPECT system interface 1124, control motors, servos, and encoders to cause gamma cameras 1104a, 1104b to rotate along gantry 1102 and to acquire two-dimensional emission data (i.e., projection images) 1132 at defined imaging positions during the rotation.

Control program 1131 may further be executed to reconstruct images 1133 from projection images 1132. Superellipse parameter values 1134 may be derived from reconstructed images 1133 as described herein.

Terminal 1140 may comprise a display device and an input device coupled to terminal interface 1125 of system 1120. Terminal 1140 may receive and display images 1133 and reference images 1134, as well as a user interface to assist comparisons therebetween. In some embodiments, terminal 1140 is a separate computing device such as, but not limited to, a desktop computer, a laptop computer, a tablet computer, and a smartphone.

Each component of imaging system 1100 may include other elements which are necessary for the operation thereof, as well as additional elements for providing functions other than those described herein. Each functional component described herein may be implemented in computer hardware, in program code and/or in one or more computing systems executing such program code as is known in the art. Such a computing system may include one or more processing units which execute processor-executable program code stored in a memory system.

What is claimed is:

1. A method comprising:
acquiring a three-dimensional image of an anatomical structure;
determining a plurality of slices of the three-dimensional image;
for each of the plurality of slices, fitting a model comprising a plurality of parameters to the anatomical structure in the slice to determine values of each of the plurality of parameters; and
determining a characterization of the anatomical structure based on the values of each of the plurality of parameters determined for each of the plurality of slices.

2. The method of claim 1, wherein the model is a superellipse model and the parameters comprise a semi-major axis length, a semi-minor axis length and a squareness factor.

3. The method of claim 1, wherein fitting the model comprises determining locations of peak intensity corresponding to the anatomical structure in the slice and fitting the model to the locations of peak intensity.

4. The method of claim 3, wherein determining the locations of peak intensity comprises:
for each of a plurality of one-dimensional lines through a center of the anatomical structure in the slice:
determining an intensity profile along the line;
determining two peaks on the intensity profile; and
determining locations of the two peaks.

5. The method of claim 4, wherein determining locations of the two peaks comprises:
fitting a Gaussian function to each of the two peaks; and
determining a location of a peak of each Gaussian function.

6. The method of claim 5, further comprising:
determining a symmetric window about each of the two peaks,
wherein fitting a Gaussian function to each of the two peaks comprises fitting a Gaussian function to pixel intensity values of each symmetric window.

7. The method of claim 6, wherein fitting a Gaussian function to each of the two peaks comprises weighting intensity values of pixels located closer to a peak higher than intensity values of pixels located farther from a peak.

8. The method of claim 1, wherein the determined characterization of the anatomical structure comprises an indication of abnormality.

9. The method of claim 1, wherein the determined characterization of the anatomical structure comprises an indication of excessive motion.

10. A system comprising:
an imaging system to acquire a three-dimensional image of an anatomical structure;
a control system to:
determine a plurality of slices of the three-dimensional image;
for each of the plurality of slices, fit a model comprising a plurality of parameters to the anatomical structure in the slice to determine values of each of the plurality of parameters; and
determine a characterization of the anatomical structure based on the values of each of the plurality of parameters determined for each of the plurality of slices.

11. The system of claim 10, wherein the model is a superellipse model and the parameters comprise a semi-major axis length, a semi-minor axis length and a squareness factor.

12. The system of claim 10, wherein fitting of the model comprises determination of locations of peak intensity corresponding to the anatomical structure in the slice and fit of the model to the locations of peak intensity.

13. The system of claim 12, wherein determination of the locations of peak intensity comprises:
for each of a plurality of one-dimensional lines through a center of the anatomical structure in the slice:
determination of an intensity profile along the line;
determination of two peaks on the intensity profile; and
determination of locations of the two peaks.

14. The system of claim 13, wherein determination of locations of the two peaks comprises:
fitting of a Gaussian function to each of the two peaks; and
determination of a location of a peak of each Gaussian function.

15. The system of claim 14, the control system further to:
determine a symmetric window about each of the two peaks,
wherein fitting of a Gaussian function to each of the two peaks comprises fitting of a Gaussian function to pixel intensity values of each symmetric window.

16. The system of claim 15, wherein fitting of a Gaussian function to each of the two peaks comprises weighting of intensity values of pixels located closer to a peak higher than intensity values of pixels located farther from a peak.

17. The system of claim 10, wherein the determined characterization of the anatomical structure comprises an indication of abnormality.

18. The system of claim 10, wherein the determined characterization of the anatomical structure comprises an indication of excessive motion.

19. One or more computer-readable media storing program code executable by one or more processing units of a computing system to cause the computing system to perform operations comprising:
acquiring a three-dimensional image of an anatomical structure;
determining a plurality of slices of the three-dimensional image;
for each of the plurality of slices, fitting a model comprising a plurality of parameters to the anatomical structure in the slice to determine values of each of the plurality of parameters; and
determining a characterization of the anatomical structure based on the values of each of the plurality of parameters determined for each of the plurality of slices.

20. The one or more computer-readable media of claim 19, wherein fitting the model comprises determining locations of peak intensity corresponding to the anatomical structure in the slice and fitting the model to the locations of peak intensity, and
wherein determining the locations of peak intensity comprises:
for each of a plurality of one-dimensional lines through a center of the anatomical structure in the slice:
determining an intensity profile along the line;
determining two peaks on the intensity profile; and
determining locations of the two peaks.
